(19) United States
(12) Patent Application Publication
(10) Pub. No.: US 2008/0269550 A1
(43) Pub. Date: Oct. 30, 2008

(54) Cardiac Apical Suction Device for Cardiac Surgery

(75) Inventor: John T. M. Wright, Denver, CO (US)

Correspondence Address: SWANSON & BRATSCHUN, L.L.C., 8210 SOUTHPARK TERRACE, LITTLETON, CO, 80120, US

(73) Assignee: Genesee BioMedical, Inc., Denver, CO (US)

(21) Appl. No.: 11/574,273

(22) PCT Filed: Aug. 26, 2005
(86) PCT No.: PCT/US05/30424

§ 371 Date: Sep. 12, 2007

Related U.S. Application Data
(60) Provisional application No. 60/604,869, filed on Aug. 26, 2004.

(51) Int. Cl. A61B 17/00

(52) U.S. Cl. 600/37

(57) ABSTRACT

A cardiac apical suction device (CASD) for supporting an apical region of a beating heart during off-pump, beating heart surgery. The apparatus consists of an outer frame configured to fit over the apical region of the heart having a flexible inner member. The inner member has an inner surface defining a cavity of select volume sized to receive the apical region of a heart. The inner member further has a peripheral lip sized to make sealing engagement along a contour of the surface of the heart. The inner member and outer frame are connected by an actuation member operative to selectively increasing the volume of the inner cavity. The CASD may be placed over the apical region of a heart such that the peripheral lip forms a sealing engagement with the surface of the heart. Upon operation of the actuation member, the increase in the volume of the inner cavity causes a decrease in the pressure within the inner member, resulting in suction adhesion between the CASD and the surface of the heart.

### FIELD OF THE INVENTION

The present invention is directed toward cardiac surgical apparatus, and more particularly toward a suction device for positioning, lifting, orienting, and supporting a beating heart during cardiac surgery.

### BACKGROUND OF THE INVENTION

Historically, coronary artery bypass surgery has been performed with the patient on a cardiopulmonary bypass machine and the heart stopped during surgery. With the heart stopped, the cardiopulmonary bypass machine circulates the patient's blood throughout his body. Surgery is performed on an exposed and still heart. Many patients poorly tolerate placement on a cardiopulmonary bypass machine. In addition, certain time and cost advantages can be achieved by eliminating the heart-lung bypass machine. Thus, an increasing number of coronary artery bypass surgeries are being performed off-pump, without a cardiopulmonary bypass machine, and with blood flow being provided by the patient's own beating heart during surgery.

During off-pump beating heart coronary artery bypass surgery, it is advantageous to place the patient in a steep Trendelenburg position and to elevate the apex of the left ventricle. Various devices are known in the prior art which facilitate the support and elevation of the left ventricle. Typically, these devices attach to and support the apex of the heart (the portion of the heart over the left ventricle and distal to the aortic arch) by means of a suction cup device.

Representative prior art devices include the devices to perform off-pump beating heart coronary bypass surgery disclosed in Spence et al., U.S. Pat. Nos. 6,019,722; 6,338,712; and 6,705,988. The Spence devices include, among other elements, a suction device for positioning near the apical region of the heart. The suction device of Spence is designed to be connected in fluid communication with an external suction source, typically the suction system readily available in typical operating rooms.

D'Arrigo, published U.S. Application Serial Number 2004/0002632, discloses a suction device including a suction cup which is optimized to minimize injury to tissue surfaces, and which is suitable for attachment to the apical region of the heart. Like Spence, the D′Arrigo device is designed for connection in fluid communication to the external suction system of a typical operating room.

Typically, the prior art features a cup which forms a suction seal with the heart tissue around an exterior rim of the cup. For example, Spence, U.S. Pat. No. 6,338,712, discusses numerous formats of suction cups which feature a first chamber having a flexible rim for engaging the tissue of the heart. Spence further discloses a second chamber separated from the first by a mesh which prevents heart tissue from being drawn into the suction opening. However, the Spence and other prior art suction cups seal along one contour of the heart tissue, typically around the exterior perimeter of the suction cup. Thus, the prior art devices can fail if either the external suction source fails or the seal between the external perimeter of the suction cup and the heart tissue is broken or released in one place.

The present invention is directed toward overcoming one or more of the problems discussed above.

### SUMMARY OF THE INVENTION

One aspect of the present invention is an apparatus for supporting a beating heart including an engagement member configured to make a substantially tight sealing engagement with the surface of the heart. The apparatus also includes an actuation member associated with an engagement member which is configured to deform the engagement member to cause suction adhesion between the engagement member and the surface of the heart. Deformation of the engagement member can occur in any manner including folding, compressing, stretching or moving a first portion of the engagement member with respect to a second portion of the engagement member.

The actuation member will typically be a mechanical device configured to deform the engagement member such as a lever, a cam or a spring plunger. The apparatus may further include at least one contact ring associated with the engagement member.

In an alternative embodiment of this aspect of the invention, the apparatus may further include an attachment structure associated with the engagement member and a support connected to the engagement member by the attachment structure. The support may be an adjustable arm connected to a rigid fixture. In certain embodiments a flexible member may be operatively disposed between the adjustable arm and the rigid fixture permitting movement of the engagement member in multiple planes relative to the rigid fixture.

Another aspect of the present invention is a cardiac apical suction device (CASD) for supporting the apical region of a beating heart during off-pump, beating heart surgery including an outer frame configured to fit over the apical region of the heart and a flexible inner member. The inner member has an inner surface defining a cavity of select volume sized to receive the apical region of a heart. The inner member further has an opening sized to make sealing engagement along a contour of the surface of the heart. The inner member and outer frame are connected by an actuation member operative to selectively increase or decrease the volume of the inner cavity. The CASD may be placed over the apical region of a heart such that the opening forms a sealing engagement with the surface of the heart. Upon operation of the actuation member, the increase in the volume of the inner cavity causes a decrease in the pressure within the inner member, resulting in suction adhesion between the CASD and the surface of the heart.

The inner surface of the inner member may be molded or formed into a peripheral lip and or more contact rings which define independent vacuum sections. Thus, if one section breaks seal with the surface of the heart and loses pressure, the other independent sections will maintain appropriate suction adhesion.

The actuation member may include any type of mechanical or electromechanical device associated with the flexible inner member and capable of selectively increasing or decreasing the volume of the inner cavity. Representative examples of actuation members include a lever operatively associated with the outer frame and further associated with an apex of the inner member opposite the opening where the inner member is placed over the heart. This embodiment of an actuation member may further include a spring associated with the lever such that application of manual actuation force to the lever in a first direction moves the apex of the inner member closer to the opening and applies tension to the spring. Release of the manual actuation force will then allow the spring recoil force to move the lever in a second substantially opposite direction and move the apex of the inner member away from the opening.

Alternative configurations of the actuation member include, but are not limited to, a cam operatively associated with the outer frame and further associated with an apex of the inner member opposite the opening such that actuation of the cam moves the apex of the inner member away from the opening. Similarly, a spring plunger may be associated with the outer frame and operatively associated with an apex of the inner member.

This embodiment of the invention may also include an attachment structure associated with the outer frame and a support connected to the outer frame by the attachment structure. The attachment structure may be an adjustable arm connected to a rigid figure. In certain embodiments the adjustable arm may be connected to the rigid fixture with a flexible member which permits movement of the outer frame in multiple planes relative to the rigid fixture.

Another aspect of the present invention is a method of supporting an apical region of a beating heart which includes placing a cup shaped member such as the CASD or engagement member described above into contact with a portion of a beating heart. The method further includes increasing an inner volume of the cup shaped member, thus decreasing the pressure within the cup shaped member resulting in suction between the cup shaped member and the heart and supporting the cup shaped member. The increase in the inner volume of the cup shaped member may result from manual operation of an actuation member. In certain embodiments the actuation member may be operated with on hand.

Another aspect of the present invention is an apparatus for supporting a portion of a heart which includes a cup shaped engagement member, a peripheral lip associated with the engagement member and at least one contact ring associated with the engagement member. Both the peripheral lip and any contact ring are sized to make first and second sealing engagements with contours of the surface of the heart.

### BRIEF DESCRIPTION OF THE DRAWINGS

FIG. 1 is a cross sectional view of a cardiac apical suction device (CASD) in accordance with one embodiment of the present invention;

FIG. 2 is a cross sectional view of the CASD of FIG. 1 illustrating the configuration when manual actuation pressure is applied to the lever;

FIG. 3 is a side view of the embodiment of the CASD illustrated in FIGS. 1-2;

FIG. 4 is a plan view of the embodiment of the CASD illustrated in FIGs.

FIG. 5 is a cross sectional view of another embodiment of the CASD;

FIG. 6 is a cross sectional view of the embodiment of FIG. 5 with the plane of view rotated 90°;

FIG. 7 is a cross sectional view of the CASD of FIG. 5 illustrating the CASD in an actuated state;

FIG. 8 is a cross sectional view of another embodiment of the CASD; and

FIG. 9 is an isometric view of an embodiment of a CASD attached to an arm attached to a sternal retractor.

### DETAILED DESCRIPTION OF THE PREFERRED EMBODIMENT

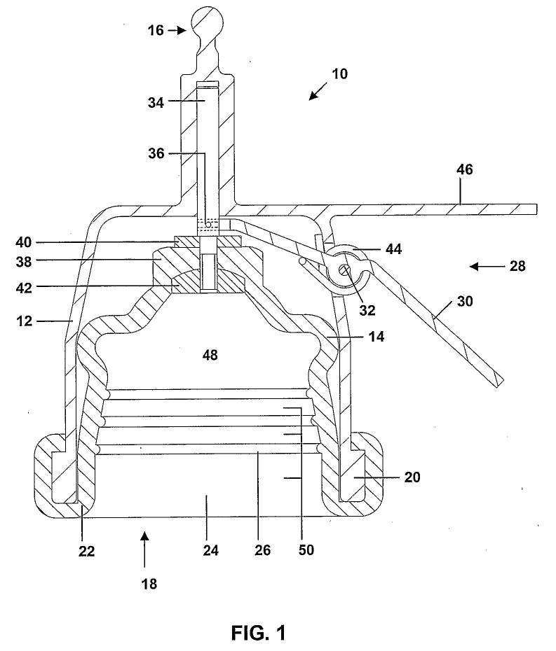

FIG. 1 is a cross sectional view of a cardiac apical suction device (CASD) 10 in accordance with one embodiment of the present invention. In this embodiment, the CASD 10 features a rigid outer frame 12 which is typically cup shaped, shaped as a truncated cone, or hemispherical. In this embodiment, the rigid outer frame is attached to a flexible inner member 14. It is important to note that the two part structure of a rigid outer frame 12 connected to a flexible inner member 14 is not limiting. For example, the CASD 10 may be an engagement member of any nature sized to make substantially airtight sealing engagement with a surface of the apical region of a heart. Thus, the CASD 10 may be a simple engagement member which is configured similarly to the flexible inner member 14 of FIG. 1, but without the additional support of an outer frame 12.

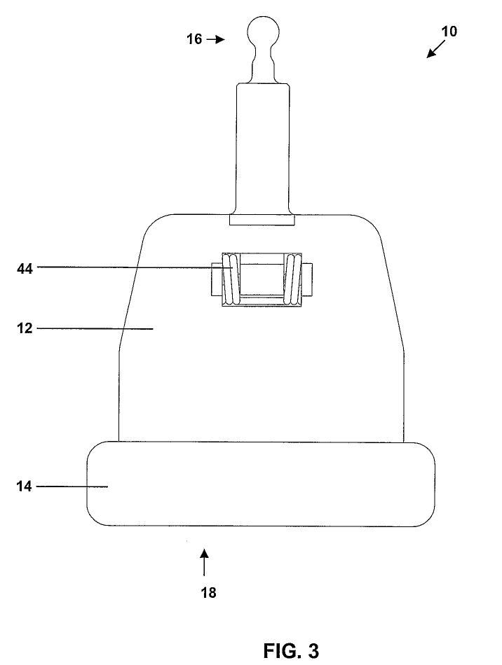

The outer frame 12 may be perforated with openings, fabricated from a rigid mesh or substantially un-perforated as is shown in FIG. 3. In any configuration the outer frame 12 will define an interior space containing a flexible inner member 14. The outer frame 12 is preferably constructed of a hard polymer or inert metal that provides for a fine finished surface, a high degree of rigidly, and good resistance to ethylene oxide, gamma radiation or other chemicals. The inner member 14 is preferably constructed of a flexible material such as an silicone elastomeric polymer which offers high flexibility, ductility, a good coefficient of friction, biocompatibility and good resistance to ethylene oxide, gamma radiation, or other compounds used for sterilization.

The CASD 10 is configured with an attachment structure 16 at one end of the outer frame 12. The opposite end of the CASD 10 features an opening 18 sized to receive and form a sealing engagement with the apical region of a beating heart. At or near the opening 18, the outer frame 12 is formed into a slot or protrusion to removably engage the inner member 14. This structure is shown on FIG. 1 as a shoulder 20 for engagement with a circular pocket formed in the inner member 14 near the opening 18. The portion of the inner member 14 at the opening 18 defines a peripheral lip 22. The peripheral lip 22 is sized to make a sealing engagement along a contour of the surface of the apical region of a heart. The inner member 14 has an inner surface 24 which may be molded or otherwise formed into one or more contact rings 26, the contact rings 26 may be sized to make secondary sealing engagement with other distinct contours of the surface of the apical region of a heart. The contact rings 26 may have triangular, hemispherical or other cross sectional shape.

The CASD 10 includes an actuation member 28 operatively associated with both the outer frame 12 and the inner member 14. The actuation member 28 shown in FIG. 1 consists of a lever 30 connected to a pivot 32 operatively associated with the outer frame 12. The lever 30 is further connected to a shaft 34 received in sliding, piston-type engagement with the outer frame 12. The connection between the lever 30 and the shaft 34 has a secondary sliding pivot 36 such that actuation of the lever 30 causes the shaft 34 to move axially with respect to the outer frame 12. The shaft 34 is connected to the inner member 14 at an apex 38 of the inner member 14. FIG. 1 depicts an embodiment in which the connection to the apex 38 of the inner member 14 is accomplished by permanently clamping the inner member 14 to the shaft 34 with reinforcement members 40, 42. Other connection methods may be employed, including but not limited to the use of a bolt head, nut and washer or any other mechanical or adhesive bonding method between the shaft 34 and the apex 38 of the inner member 14. For all embodiments it is critical that the connection between the actuation member 28 and inner member 14 be substantially airtight, allowing a suction engagement with the apical region of a heart to be maintained during a surgical procedure.

In the embodiment illustrated in FIG. 1, the actuation member 28 also features a spring 44 operatively associated with the lever 30 so that manual actuation of the lever 30 applies tension to the spring 44. Similarly, the release of manual actuation force from the lever 30 allows spring 44 recoil forces to return the lever 30 to the start position shown in FIG. 1. The spring 44 is depicted herein as a coil spring, but the spring 44 can be implemented with a compression, extension, leaf or other type of spring in addition to the recoil potential of the elastic member 14. In the embodiment shown in FIG. 1, a portion of the outer frame 12 is formed into a handle 46 which is sized similarly to the lever 30 and oriented such that the lever 30 can be brought to a position parallel with the handle 46.

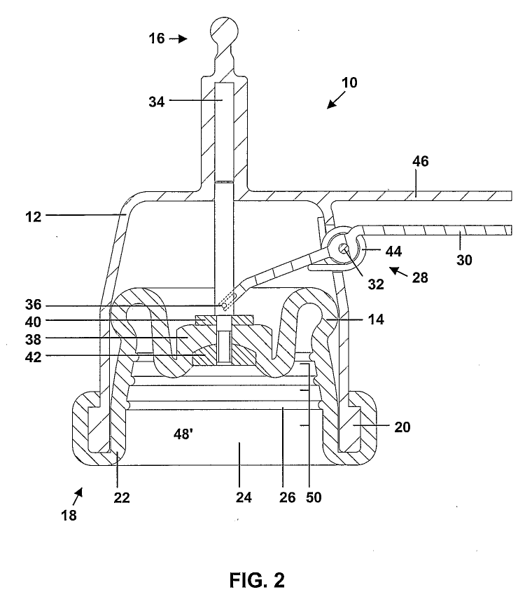

FIG. 1 illustrates the CASD 10 in a state where no manual actuation force is being applied to the lever 30. In this state, the inner member 14 defines a relatively large interior volume 48. FIG. 2 illustrates the same embodiment of the CASD 10 with manual actuation pressure applied to the lever 30. Upon the application of manual actuation pressure to the lever 30, the shaft 34 is moved linearly toward the opening 18 with respect to the outer frame 12. Accordingly, the shaft 34 depresses the apex 38 of the inner member 14 toward the opening 18, resulting in a relatively decreased interior volume 48′ within the inner member 14. In the state shown in FIG. 2, the spring 44 is under tension from the application of manual actuation pressure, and the inner member 14 is elastically collapsed.

In another embodiment, the transition form a relatively large interior volume 48 to the relatively decreased interior volume 48′ can be accomplished by stretching a surface of the inner member, thus accomplishing the volume change without folding or collapsing of the inner member 14.

The CASD 10 may be used by a surgeon to support the apical region of a heart during off-pump beating heart surgery. To apply the CASD 10 embodiment of FIG. 1 to the apical region of a patient's heart, the surgeon first attaches the CASD 10 to a support structure, for example an arm clamped to a sternal retractor, and then applies manual actuation pressure to the lever 30, placing the CASD 10 into the state shown in FIG. 2. The surgeon then places the CASD 10 over the apical region of the patient's heart such that the heart is received within the opening 18 of the CASD 10 and the peripheral lip 22 contacts a contour of the surface of the heart in a substantially airtight engagement. With the CASD 10 thus snugly placed, the surgeon may release manual actuation pressure. The recoil force of the spring 44 will then be applied to the lever 30 and thereby to the shaft 34, moving the shaft 34 in a linear fashion away from the opening 18 with respect to the outer frame 12, assisted by the relaxation of the inner member 14. Consequently, the apex 38 of the inner member 14 is moved toward the state shown in FIG. 1. The above described movement results in a relative increase in the interior volume 48 of the inner member 14. Since the peripheral lip 22 has been placed in a substantially airtight sealing engagement with a contour of the heart, the increase in the interior volume 48 will result in decreased pressure within the interior of the inner member 14 resulting in suction adhesion between the CASD 10 and the patient's heart.

It is important during beating heart surgery that suction adhesion between the CASD 10 and the apical region of the heart not be lost. Although a first sealing engagement may be made between the peripheral lip 22 and a contour of the heart, in an alternative embodiment of the invention, the inner surface 24 of the inner member 14 is formed or molded into one or more contact rings 26 which form secondary or tertiary sealing engagements with other distinct contours of the surface of the heart. Thus, the contact rings 26 divide the inner surface 24 of the inner member 14 into multiple independent sections 50. If one of the independent sections 50 loses its seal and, thus, loses suction adhesion with the surface of the heart, the other sections 50 may still maintain adhesion between the heart surface and the CASD 10.

Upon completion of surgery, the surgeon may release the CASD 10 from the apical region of the heart by reapplying manual actuation pressure to the lever 30, returning the CASD 10 to the FIG. 2 state. In this state, there is no pressure differential between the interior of the inner member 14 and the exterior of the inner member 14, so suction adhesion between the CASD 10 and the beating heart is released.

FIG. 3 is a side view of the embodiment of the CASD 10 illustrated in FIGS. 1-2. As illustrated in FIG. 3, more than one spring 44 may be associated with the lever 30 and pivot 32. The number and size of the spring(s) 44 may be selected to assure that the recoil force applied through the lever 30 to the inner member 14 combined with the elastic recoil force generated by inner member 14 is sufficient to maintain suction adhesion with the surface of a heart, but not so strong as to damage heart tissue. As is also shown on FIG. 3, the inner member 14 may wrap around and wholly enclose the outer frame 12 in the region of the shoulder 20. Thus, the only portion of the CASD 10 which is likely to contact heart tissue is the inner member 14. This configuration has several benefits, including contacting the heart only with relatively soft and flexible inner member 14 material. In addition, the inner member 14 may be replaced as needed to assure a sterile and effective contact surface with the heart.

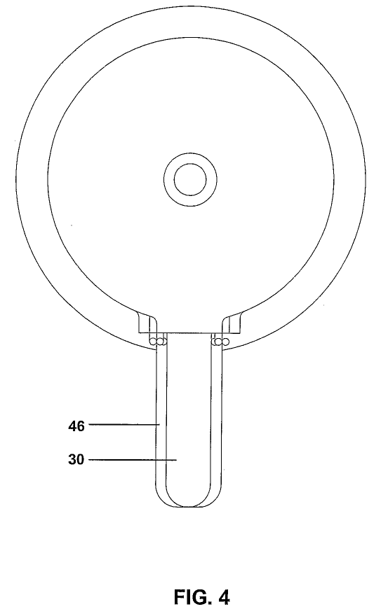

FIG. 4 is a plan view of the embodiment of the CASD 10 illustrated in FIGS. 1-3. Preferably, the handle 46 and lever 30 are sized and configured as shown in FIG. 4 so that a surgeon may easily place and remove the CASD 10 on a patient's heart using only one hand. Typically, the surgeon's other hand will be used to tighten or secure an arm or other structure holding the CASD 10 in an appropriate position.

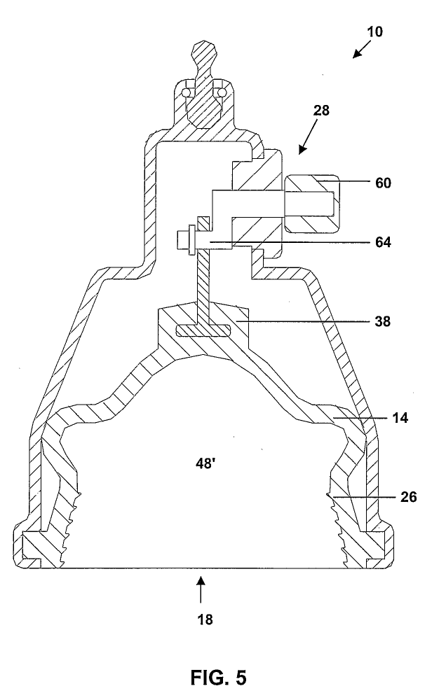

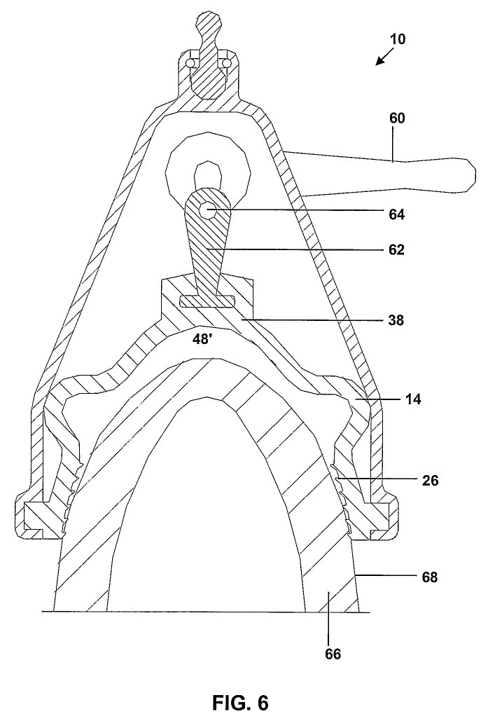

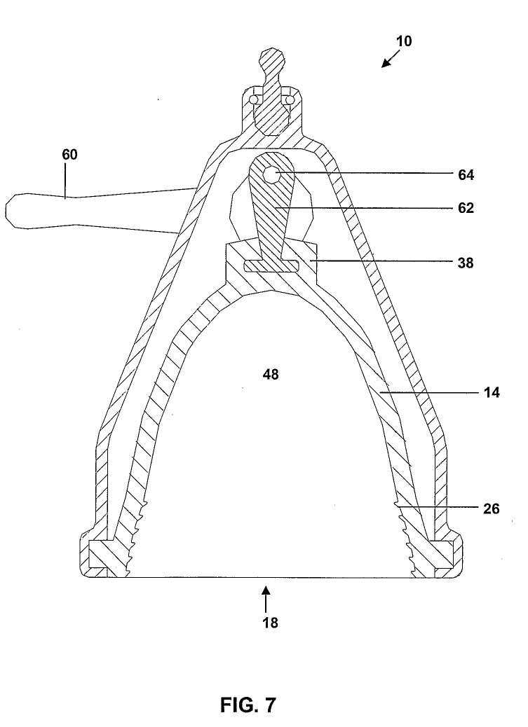

The actuation member 28 can be any mechanical or electromechanical device which can move the apex 38 of the inner member 14, thus causing an increase or decrease in the interior volume 48, 48′. An alternative actuation device is illustrated in FIGS. 5-7. FIGS. 5-7 are cutaway views along first and second orthagonal planes of an embodiment featuring a cam operated actuation member 28. The structure of the CASD 10 is substantially similar to that described above with respect to the embodiment of FIGS. 1-4, however, the lever 30 and shaft 34 are replaced with a handle 60 which is indirectly attached to the apex 38 of the inner member 14 through a linkage 62. The handle 60 and linkage 62 are attached to each other by means of an offset cam 64. Rotation of the handle 60 180° from the position shown in FIGS. 5-6 to the position shown in FIG. 7 will move the apex 38 of the inner member 14 away from the opening 18 of the CASD 10, thus increasing the interior volume 48 of the inner member 14. Also shown in FIG. 6, for illustration purposes only, is the apex of a heart 66 having a surface 68 which has been placed in sealing engagement with an embodiment of contact rings 26. The embodiment shown in FIGS. 5-7 does not feature a distinct peripheral lip. In the embodiment illustrated in FIGS. 5-7, the contact rings 26 are formed as relatively thin, triangular sections to facilitate firm suction adhesion with the surface 68 of the heart 66.

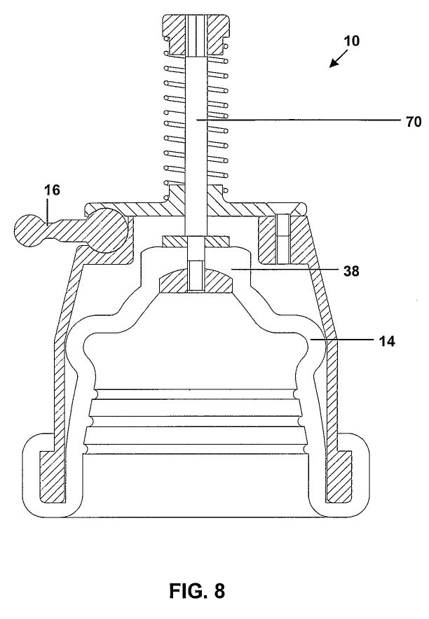

FIG. 8 shows another alternative embodiment of the CASD 10 featuring another type of articulation device. In the FIG. 8 embodiment, the attachment structure 16 is placed perpendicular to the opening 18 and movement of the apex 38 of the inner member 14 is accomplished by depressing and releasing a spring loaded plunger 70, which is bonded to the inner member 14.

Each embodiment of the CASD 10 featuring different actuation devices is equally suitable for supporting a beating heart. The embodiment shown in FIGS. 1-4 provides for the application of the CASD 10 to a heart using only one hand.

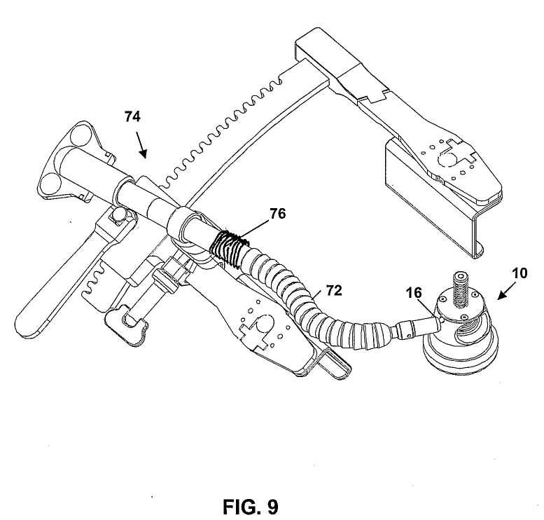

In use, the CASD 10 will be supported with respect to the patient's body. Various clamps, arms, or other mechanical devices known in the cardiac surgery arts can be used to support the CASD 10. As shown in FIG. 9, an arm 72, which may be a locking articulated arm or other type of adjustable arm, may be attached at one end to the attachment structure 16 of a CASD 10. The opposite end of the arm 72 will typically be clamped or otherwise attached to a suitably rigid fixture which is usually a sternal retractor 74. Thus, the CASD 10 may be used to lift, orient, and support a beating heart during surgery. A flexible connection 76 which may be a spring, elastomeric portion or flexible joint may be used to connect the arm 72 to the sternal retractor 76 thus allowing multiplaner movement of the CASD 10, within a limited range, as a patient's heart beats during surgery.

What is claimed is:

1. An apparatus for supporting a beating heart comprising:
an engagement member configured to make a substantially air tight sealing engagement with a surface of the heart; and
an actuation member operatively associated with the engagement member configured to deform the engagement member to cause suction adhesion between the engagement member and the surface of the heart.

2. The apparatus of claim 1 wherein the actuation member comprises at least one of a lever, a cam and a spring plunger.

3. The apparatus of claim 1 further comprising at least one contact ring operatively associated with the engagement member.

4. The apparatus of claim 1 further comprising:
an attachment structure operatively associated with the engagement member; and
a support connected to the engagement member by the attachment structure.

5. The apparatus of claim 4 wherein the support comprises:
an adjustable arm; and
a rigid fixture.

6. The apparatus of claim 5 wherein the support further comprises a flexible member operatively disposed between the adjustable arm and the rigid fixture permitting movement of the engagement member in multiple planes relative to the rigid fixture.

7. An apparatus for supporting an apical region of a beating heart comprising:
an frame configured to fit over the apical region of the heart;
a flexible inner member operatively associated with the frame with the flexible inner member comprising an inner surface defining an inner cavity of a first volume sized to receive the apical region a portion of a heart, the inner surface further comporting an opening configured to make a sealing engagement along a surface of the apical region of the heart; and
an actuation member operatively associated with the inner member expanding the inner cavity to a second volume greater than the first volume.

8. The apparatus of claim 7 wherein the actuation member comprises a lever operatively associated with the outer frame and further operatively associated with an apex of the inner member distal to the opening.

9. The apparatus of claim 8 further comprising a spring operatively associated with the lever wherein the application of a manual actuation force to the lever in a first direction moves the apex of the inner member closer to the opening and applies tension to the spring, and release of the manual actuation force allows a spring recoil force to move the lever in a second, substantially opposite direction and move the apex of the inner member away from the opening.

10. The apparatus of claim 7 further comprising at least one contact ring associated with the inner surface of the inner member sized to make a second sealing engagement with a second portion of the surface of the apical region of the heart.

11. The apparatus of claim 7 wherein the actuation member comprises a cam operatively associated with the outer frame and further operatively associated with an apex of the inner member distal to the opening such that actuation of the cam moves the apex of the inner member away from the opening.

12. The apparatus of claim 7 wherein the actuation member comprises a spring and a plunger operatively associated with the outer frame and further operatively associated with an apex of the inner member distal to the opening such that actuation of the plunger moves the apex of the inner member toward the opening.

13. The apparatus of claim 7 further comprising:
an attachment structure operatively associated with the outer frame; and
a support connected to the outer frame by the attachment structure.

14. The apparatus of claim 13 wherein the support comprises:
an adjustable arm; and
a rigid fixture.

15. The apparatus of claim 14 wherein the support further comprises a flexible member operatively disposed between the adjustable arm and the rigid fixture permitting movement of the outer frame in multiple planes relative to the rigid fixture.

16. A method of supporting an apical region of a beating heart comprising:
placing a cup shaped member into sealing engagement with the apical region of the heart;
increasing an inner volume of the cup shaped member thus decreasing the pressure within the cup shaped member resulting in suction between the cup shaped member and the apical region of the heart; and
supporting the cup shaped member.

17. The method of claim 16 wherein the increase in the inner volume of the cup shaped member results from the manual operation of an actuation member.

18. The method of claim 17 wherein the actuation member can be operated with one hand.

19. The method of claim 16 wherein supporting the cup shaped member comprises:
attaching the cup shaped member to an adjustable arm; and
attaching the adjustable arm to a rigid fixture.

20. The method of claim 19 farther comprising attaching the adjustable arm to the rigid fixture with a flexible connection.

21. An apparatus for supporting a portion of a heart comprising:
a cup shaped engagement member;
a peripheral lip associated with the engagement member sized to make a first sealing engagement along a first contour of a surface of the heart; and
at least one contact ring associated with the engagement member sized to make a second sealing engagement with a second contour of the surface of the heart.

22. The apparatus of claim 20 wherein the at least one contact ring has a triangular cross section.
